(19) United States
(12) Patent Application Publication
(10) Pub. No.: US 2012/0225949 A1
(43) Pub. Date: Sep. 6, 2012

(54) COMPOSITIONS AND METHODS FOR TREATING BIPOLAR DISORDER

(76) Inventor: Demitri Papalos, Maplewood, NJ (US)

(21) Appl. No.: 13/389,781

(22) PCT Filed: Aug. 13, 2010
(86) PCT No.: PCT/US10/45526

§ 371 Date: May 2, 2012

Related U.S. Application Data
(63) Continuation-in-part of application No. 12/541,403, filed on Aug. 14, 2009.

(51) Int. Cl. A61K 31/135; A61P 25/18

(52) U.S. Cl. 514/646

(57) ABSTRACT

The present invention is directed to a method for treating symptoms associated with bipolar disorder. The method includes administering to a subject in need of such treatment a dose of ketamine sufficient to alleviate symptoms associated with bipolar disorder, childhood-onset bipolar disorder, or childhood-onset bipolar disorder with Fear of Harm.

### BACKGROUND OF THE DISCLOSURE

Bipolar disorder, also known as manic depression, manic depressive disorder or bipolar affective disorder, is a psychiatric diagnosis that describes a category of mood disorders defined by the presence of one or more episodes of abnormally elevated mood clinically referred to as mania or, if milder, hypomania.

### SUMMARY OF THE DISCLOSURE

Disclosed herein, in certain embodiments, is a method for treating childhood-onset bipolar disorder in a subject in need thereof comprising administering a therapeutic-effective amount of ketamine. In some embodiments, the subject is under the age of 18. In some embodiments, the subject presents with Fear of Harm. In some embodiments, the subject presents with at least one of the following symptoms: mood cycling (i.e., cycling between manic episodes, depressive episodes, and normal moods), obsessive fear of harm, severe aggression, territorial aggression, thermal dysregulation, night sweats, inability to fall asleep, inability to stay asleep, disorganized speech, rapid speech, loud speech, unclear speech, unusual speech timbre, disorganized thoughts, excessive ritualization, reliance on transitional objects, hoarding, extreme separation anxiety, hallucinations, delusions, and sweet cravings. In some embodiments, the ketamine is racemic. In some embodiments, the ketamine is (R)-ketamine. In some embodiments, the ketamine is (S)-ketamine. In some embodiments, the ketamine composition is administered nasally. In some embodiments, the ketamine composition is administered orally. In some embodiments, the therapeutically-effective amount of ketamine is about 0.01 to about 1 mg/kg of body weight. In some embodiments, the therapeutically-effective amount of ketamine is about 0.05 to about 0.7 mg/kg of body weight.

Disclosed herein, in certain embodiments, is the use of a therapeutic effective amount of ketamine to treat childhood-onset bipolar disorder with Fear of Harm. In some embodiments, the subject is under the age of 18. In some embodiments, the subject presents with Fear of Harm. In some embodiments, the subject presents with at least one of the following symptoms: mood cycling (i.e., cycling between manic episodes, depressive episodes, and normal moods), obsessive fear of harm, severe aggression, territorial aggression, thermal dysregulation, night sweats, inability to fall asleep, inability to stay asleep, disorganized speech, rapid speech, loud speech, unclear speech, unusual speech timbre, disorganized thoughts, excessive ritualization, reliance on transitional objects, hoarding, extreme separation anxiety, hallucinations, delusions, and sweet cravings. In some embodiments, the ketamine is racemic. In some embodiments, the ketamine is (R)-ketamine. In some embodiments, the ketamine is (S)-ketamine. In some embodiments, the ketamine composition is administered nasally. In some embodiments, the ketamine composition is administered orally. In some embodiments, the therapeutically-effective amount of ketamine is about 0.01 to about 1 mg/kg of body weight. In some embodiments, the therapeutically-effective amount of ketamine is about 0.05 to about 0.7 mg/kg of body weight.

### DETAILED DESCRIPTION OF THE INVENTION

Disclosed herein, in certain instances, are methods of treating bipolar disorder. In some embodiments, the bipolar disorder is childhood-onset bipolar disorder. In some embodiments, the bipolar disorder is childhood-onset bipolar disorder with Fear of Harm (FOH). In some embodiments, the bipolar-disorder is childhood-onset bipolar disorder which presents with at least one of the following symptoms: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of ketamine sufficient to alleviate symptoms associated with bipolar disorder, childhood-onset bipolar disorder, or childhood-onset bipolar disorder with Fear of Harm.

Disclosed herein, in certain instances, are methods of treating bipolar disorder. In some embodiments, the bipolar disorder is childhood-onset bipolar disorder. In some embodiments, the bipolar disorder is childhood-onset bipolar disorder with Fear of Harm (FOH). In some embodiments, the bipolar-disorder is childhood-onset bipolar disorder which presents with at least one of the following symptoms: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of ketamine sufficient to alleviate symptoms associated with bipolar disorder, childhood-onset bipolar disorder, or childhood-onset bipolar disorder with Fear of Harm.

Disclosed herein, in certain instances, are methods of treating at least one symptom associated with bipolar disorder. In some embodiments, the method comprises treating at least one symptom of childhood-onset bipolar disorder presenting with Fear of Harm (FOH). In some embodiments, the at least one symptom is: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of ketamine sufficient to alleviate symptoms associated with bipolar disorder, childhood-onset bipolar disorder, or childhood-onset bipolar disorder with Fear of Harm.

### CERTAIN DEFINITIONS

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth.

The terms “individual,” “subject,” or “patient” are used interchangeably. As used herein, they mean any mammal (i.e. species of any orders, families, and genus within the taxonomic classification animalia: chordata: vertebrata: mammalia). In some embodiments, the mammal is a human. In some embodiments, the mammal is a non-human. In some embodiments, the mammal is a member of the taxonomic orders: primates (e.g. lemurs, lorids, galagos, tarsiers, monkeys, apes, and humans); rodentia (e.g. mice, rats, squirrels, chipmunks, and gophers); lagomorpha (e.g. hares, rabbits, and pika); erinaceomorpha (e.g. hedgehogs and gymnures); soricomorpha (e.g. shrews, moles, and solenodons); chiroptera (e.g., bats); cetacea (e.g. whales, dolphins, and porpoises); carnivora (e.g. cats, lions, and other feliformia; dogs, bears, weasels, and seals); perissodactyla (e.g. horse, zebra, tapir, and rhinoceros); artiodactyla (e.g. pigs, camels, cattle, and deer); proboscidea (e.g. elephants); sirenia (e.g. manatees, dugong, and sea cows); cingulata (e.g. armadillos); pilosa (e.g. anteaters and sloths); didelphimorphia (e.g. american opossums); paucituberculata (e.g. shrew opossums); microbiotheria (e.g. Monito del Monte); notoryctemorphia (e.g. marsupial moles); dasyuromorphia (e.g. marsupial carnivores); peramelemorphia (e.g. bandicoots and bilbies); or diprotodontia (e.g. wombats, koalas, possums, gliders, kangaroos, wallaroos, and wallabies). In some embodiments, the animal is a reptile (i.e. species of any orders, families, and genus within the taxonomic classification animalia: chordata: vertebrata: reptilia). In some embodiments, the animal is a bird (i.e. animalia: chordata: vertebrata: ayes). None of the terms require or are limited to situation characterized by the supervision (e.g. constant or intermittent) of a health care worker (e.g. a doctor, a registered nurse, a nurse practitioner, a physician's assistant, an orderly, or a hospice worker).

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In some embodiments, the effective amount is a dose that is generally effective in alleviating, reducing, noticeably reducing, or eliminating, symptoms associated with bipolar disorder or mania. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering,” “administration,” and the like, as used herein, refer to the methods that are used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In some embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material is administered to an individual without causing significant undesirable biological effects or significantly interacting in a deleterious manner with any of the components of the composition in which it is contained.

### Bipolar Disorder

Disclosed herein, in certain instances, are methods of treating bipolar disorder. In some embodiments, the bipolar disorder is childhood-onset bipolar disorder. In some embodiments, the bipolar disorder is childhood-onset bipolar disorder with Fear of Harm (FOH). In some embodiments, the bipolar-disorder is childhood-onset bipolar disorder which presents with at least one of the following symptoms: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of ketamine sufficient to alleviate symptoms associated with bipolar disorder, childhood-onset bipolar disorder, or childhood-onset bipolar disorder with Fear of Harm.

Disclosed herein, in certain instances, are methods of treating at least one symptom associated with bipolar disorder. In some embodiments, the method comprises treating at least one symptom of childhood-onset bipolar disorder presenting with Fear of Harm (FOH). In some embodiments, the at least one symptom is: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of ketamine sufficient to alleviate symptoms associated with bipolar disorder, childhood-onset bipolar disorder, or childhood-onset bipolar disorder with Fear of Harm.

As used herein, a “bipolar disorder” means a disorder characterized by unusually intense emotional states that occur in distinct periods called “mood episodes.” An overly elated or overexcited state is called a manic episode, and an extremely sad or hopeless state is called a depressive episode. Individuals who experience manic episodes also commonly experience depressive episodes or symptoms, or mixed episodes in which features of both mania and depression are present at the same time. These episodes are usually separated by periods of “normal” mood, but in some individuals, depression and mania may rapidly alternate, known as rapid cycling. Extreme manic episodes can sometimes lead to psychotic symptoms such as delusions and hallucinations. Patients affected by bipolar disorder have had at least one manic or hypomanic (mild mania) episode. Patients with full manias and depression are indicated as having “bipolar I disorder”. Patients with hypomanias and depressions are described as having “bipolar II disorder.” Onset of episodes tends to be acute, with symptoms developing over days to weeks.

Symptoms of mania or a manic episode include both mood changes and behavioral changes. Mood changes include the following: a long period of feeling “high,” or an overly happy or outgoing mood; and extremely irritable mood, agitation, feeling “jumpy” or “wired.” Behavioral Changes include the following: talking very fast, jumping from one idea to another, having racing thoughts; being easily distracted; increasing goal-directed activities, such as taking on new projects; being restless; sleeping little; having an unrealistic belief in one's abilities; behaving impulsively and taking part in a lot of pleasurable; and high-risk behaviors, such as spending sprees, impulsive sex, and impulsive business investments.

Symptoms of depression or a depressive episode include both mood changes and behavioral changes. Mood changes include the following: a long period of feeling worried or empty; and loss of interest in activities once enjoyed, including sex. Behavioral Changes include the following: feeling tired or “slowed down”; having problems concentrating, remembering, and making decisions; being restless or irritable; changing eating, sleeping, or other habits; and thinking of death or suicide, or attempting suicide.

### Childhood-Onset Bipolar Disorder

It was traditionally believed that bipolar disorder could, at the earliest, develop in a person's late teens or early adult. Indeed, many parents are told that the diagnosis cannot be made until the child grows into the upper edges of adolescence—between 16 and 19 years old. Bipolar disorder, however, also affects close to 1 million children and adolescents in the United States at any given time. A proper diagnosis of childhood-onset (or early-onset) bipolar disorder may be made in a child as early as early as 18 months. As used herein, “childhood-onset bipolar disorder” means bipolar disorder in a human being under the age of 18 years old.

In some embodiments, childhood-onset bipolar disorder is detected using any method known in the art. In some embodiments, childhood-onset bipolar disorder is detected by use of the Childhood Bipolar Questionaire (CBQ).

Bipolar disorder manifests differently in children or adolescents as compared to adults. Adults experience abnormally intense moods for weeks or months at a time, but children appear to experience rapid shifts of mood that they commonly cycle many times within the day. This cycling pattern associated with low arousal states in the mornings followed by afternoons and evenings of increased energy.

In some embodiments, an individual suffering from childhood-onset bipolar disorder demonstrates the behavioral phenotype Fear of Harm (FOH). The FOH phenotype has been associated with children with childhood-onset bipolar disorder with increased mania and depression and other indices which demonstrate increased severity of illness. In some embodiments, subjects are tested for childhood-onset bipolar disorder and for FOH. In some embodiments, subjects are chosen for treatment according to a method disclosed herein because they have been diagnosed with childhood-onset bipolar disorder and are FOH positive.

### Fear of Harm (FOH)

The FOH phenotype is a clinically homogeneous behavioral phenotype of childhood-onset bipolar disorder with early age of onset, severe manic and depressive symptoms, early and frequent psychiatric hospitalizations, significant social impairment and school problems. The symptoms that define the FOH subtype include Territorial Aggression, Harm to Self and Others, Self-esteem, and Psychosis/Parasomnias/Sweet Craving/Obsessions (PPSO). The PPSO factor comprises a unique cluster of symptoms that includes psychosis, parasomnias (enuresis and night terrors), craving for sweets, food hoarding and contamination fears. Individuals with FOH often experience sleep-onset delay, sleep fragmentation and morning sleep inertia. Temperature/actigraphy studies of childhood-onset bipolar disorder suggest that one of the features of the condition is a delay of circadian sleep rhythms and some disturbance in thermoregulation that would produce symptoms of initial insomnia and sleep inertia (sleep onset and sleep offset). Also, data from children with the FOH phenotype suggest that there is a circadianphase delay in sleep timing and temperature dysregulation at sleep onset. Difficulty arising in the AM (sleep inertia), settling at night, getting to sleep and sleeping fitfully or awakening in the middle of the night constitute sleep initiation and maintenance problems that specifically characterize the FOH Sleep/Arousal factor. In addition, arousal parasomnias, including enuresis, hypnogogic and hypnopompic hallucinations, night terrors and vivid nightmares—often containing images of gore and mutilation, themes of pursuit, bodily threat and parental abandonment are features of the PPSO factor. Taken together this set of symptoms is indicative of both primary sleep problems and sleep pertubations secondary to altered circadian and ultradian rhythms of sleep, wakefulness and temperature. Disturbances in areas that regulate these rhythms would likely result in difficulties with transitions from sleep to waking, waking to sleep and between REM and NREM sleep phases.

In certain instances, dysfunction of orexigenic neurons (and, the orexigenic neuropeptide circuit) results in the FOH phenotype. Evidence indicates that hypothalamic preoptic area orexigenic neurons are active during sleep and in response to the increase in homeostatic pressure for sleep. They orchestrate onset, offset and maintenance of sleep as well as the regulation of REM/nonNREM sleep transitions by inhibitory modulation of multiple arousal systems. Disruption of the orexin system results in human narcolepsy, characterized by excessive daytime sleepiness, premature transitions to REM sleep (sleep-onset REM), and cataplexy. In addition, orexin neurons are involved in thermoregulation (the ability to dissipate heat efficiently at night is permissive of sleep onset, and the capacity to conserve heat efficiently in the morning reduces sleep inertia and promotes wakefulness). Evidence also suggests that orexin neurons regulate appetite, including food foraging and hoarding (appetitive behavior) and food intake (consummatory behavior). Orexin neurons may also affect territorial aggression and fear arousal.

Current Methods of Treatment Treatment of bipolar disorder can be problematic. In adults, mania requires prompt treatment because it can rapidly worsen, resulting in poor judgment that endangers interpersonal relationships, jobs, and finances. Management is founded upon medication, provision of a low-stimulation environment, and protecting the patient from undertaking potentially harmful activities. Initial management of acute mania is often best accomplished through hospitalization. Thus, the management of bipolar disorder can be expensive, intrusive, and difficult. In addition, despite the now routine use of maintenance treatment for bipolar disorder, up to 90 percent of patients experience at least one relapse within 5 years of their original diagnosis.

Adult bipolar disorder treatments generally include one or more of the following: mood stabilizers; antidepressants; antipsychotics; and electroconvulsive therapy (ECT). There are largely three main types of drugs used for the treatment of bipolar disorder. These are lithium, anticonvulsants (e.g., Depakote or other valproate products) and atypical neuroleptics (e.g., risperidone, olanzapine, ziprasidone, aripiprazole, and quetiapine). These treatments may be used to treat both adults and children.

Children, remain difficult to treat. Many children will not respond well to or are refractory to treatments designed for adult-like symptoms or manifestation of the disorder. The currently used therapeutic agents often cause negative side-effects in children. Side-effects include the following. Atypical neuroleptics (except aripiprazole) are associated with marked weight gain in many children. The dangers of this weight gain include glucose problems that may include the onset of diabetes and increased blood lipids that may worsen heart and stroke problems later in life. In addition, these drugs can cause an illness called tardive dyskinesia, which involves repetitive movements including unsightly, repeated movements of the tongue in and out of the mouth or cheek. Depakote may also be associated with increased weight and possibly with a disease called polycystic ovarian syndrome (POS). In some cases POS is associated with infertility later in life. Lithium has been on the market the longest and is the only medication that has been shown to be effective against future episodes of mania, depression and completed suicides. Some people who take lithium over a long time will need a thyroid supplement and in rare cases may develop serious kidney disease.

Thus, there is a need for improved methods and compositions for the diagnosis and treatment of bipolar disorder, including childhood-onset bipolar disorder and childhood-onset bipolar disorder with FOH.

### Ketamine

Disclosed herein, in certain instances, are methods of treating bipolar disorder. In some embodiments, the bipolar disorder is childhood-onset bipolar disorder. In some embodiments, the bipolar disorder is childhood-onset bipolar disorder with Fear of Harm (FOH). In some embodiments, the bipolar-disorder is childhood-onset bipolar disorder which presents with at least one of the following symptoms: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of ketamine sufficient to alleviate symptoms associated with bipolar disorder, childhood-onset bipolar disorder, or childhood-onset bipolar disorder with Fear of Harm.

Disclosed herein, in certain instances, are methods of treating at least one symptom associated with bipolar disorder. In some embodiments, the method comprises treating at least one symptom of childhood-onset bipolar disorder presenting with Fear of Harm (FOH). In some embodiments, the at least one symptom is: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of ketamine sufficient to alleviate symptoms associated with bipolar disorder, childhood-onset bipolar disorder, or childhood-onset bipolar disorder with Fear of Harm.

As used herein, the term “ketamine” refers to ketamine (i.e., [(2-2-chlorophenyl)-2-(methylamino)-cyclohexanone]), metabolites of ketamine (e.g., norketamine), pharmaceutically acceptable salts thereof (e.g., ketamine tannate, ketamine maleate, ketamine hydrochloride), and biologically equivalent derivatives and analogs thereof (e.g., ketamine aspartate and ketamine succinate). Also included within the scope of the term “ketamine,” are isomers and enantiomers thereof.

Ketamine is a chiral compound. The R and S stereoisomers have different binding affinities: (S)-ketamine has about four times greater affinity for the PCP site of the NMDA receptor than does (R)-ketamine. (S)-ketamine induces drowsiness more strongly than the (R) enantiomer. (R)-ketamine is a hallucinogenic.

Ketamine affects multiple biological pathways. In certain instances, ketamine is an non-competitive NMDA receptor antagonist. Ketamine also affect multiple other biological pathways. In certain instances, ketamine binds to opioid μ receptors and sigma receptors. In certain instances, ketamine is a D2 receptor partial agonist. In certain instances, ketamine is a dopamine reuptake inhibitor. In certain instances, ketamine inhibits hyperpolarization-activated cyclic nucleotide-modulated (HCN1) cation channels, which mediate the “sag” current in neurons. In certain instances, (rac)-ketamine acts as a noncompetitive inhibitor of the α7 nAChR.

### NMDA Receptor Antagonists

Disclosed herein, in certain instances, are methods of treating bipolar disorder. In some embodiments, the bipolar disorder is childhood-onset bipolar disorder. In some embodiments, the bipolar disorder is childhood-onset bipolar disorder with Fear of Harm (FOH). In some embodiments, the bipolar-disorder is childhood-onset bipolar disorder which presents with at least one of the following symptoms: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of an NMDA receptor antagonist sufficient to alleviate symptoms associated with bipolar disorder, childhood-onset bipolar disorder, or childhood-onset bipolar disorder with Fear of Harm. In some embodiments, the NMDA receptor antagonist targets the phencyclidine site of the NMDA receptor.

Disclosed herein, in certain instances, are methods of treating at least one symptom associated with bipolar disorder. In some embodiments, the method comprises treating at least one symptom of childhood-onset bipolar disorder presenting with Fear of Harm (FOH). In some embodiments, the at least one symptom is: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of an NMDA receptor antagonist sufficient to alleviate symptoms associated with bipolar disorder, childhood-onset bipolar disorder, or childhood-onset bipolar disorder with Fear of Harm. In some embodiments, the NMDA receptor antagonist targets the phencyclidine site of the NMDA receptor.

The NMDA receptor (NMDAR) is an ionotropic glutamate receptor. Activation of the NMDA receptor enables the transfer of electrical signals between neurons in the brain and in the spinal column. Activation of NMDA receptors results in the opening of an ion channel that is nonselective to cations (e.g., Ca²⁺, Na⁺, and K⁺). NMDA ion channels open when the following two conditions are met simultaneously: glutamate is bound to the receptor, and the postsynaptic cell is depolarized (which removes the Mg²⁺ blocking the channel). The excitatory postsynaptic potential (EPSP) produced by activation of an NMDA receptor increases the concentration of Ca²⁺ in the cell. Ca²⁺ functions as a second messenger in various signaling pathways. The NMDA receptor therefore functions as a “molecular coincidence detector”.

The NMDA receptor forms a heterotetramer between two NR1 and two NR2 subunits; two obligatory NR1 subunits and two regionally localized NR2 subunits. Each receptor subunit has modular design. The NR1 subunits bind the co-agonist glycine and NR2 subunits bind the neurotransmitter glutamate.

In some embodiments, the NMDA receptor antagonist binds to the NMDA receptor. In some embodiments, the NMDA receptor antagonist binds to an NR1 subunit. In some embodiments, the NMDA receptor antagonist binds to an NR2 subunit. In some embodiments, the NMDA receptor antagonist binds to an NR3 subunit. In some embodiments, the NMDA receptor antagonist binds to the phencyclidine site of the NMDA receptor.

In some embodiments, the NMDA receptor antagonist is: 1-aminoadamantane; dextromethorphan; dextrorphan; ibogaine; ifenprodil; (S)-ketamine; (R)-ketamine; dizocilpine (MK-801); gacyclidine; traxoprodil; D-2-amino-5-phosphonopentanoic acid (D-AP5); 3-((±)2-carboxypiperazin-4-yl)-propyl-1-phosphonic acid (CPP); conantokin; 7-chlorokynurenate (7-CK); licostinel; nitrous oxide; phencyclidine; riluzole; tiletamine; aptiganel; remacimide; DCKA (5;7-dichlorokynurenic acid); kynurenic acid; 1-aminocyclopropanecarboxylic acid (ACPC); AP7 (2-amino-7-phosphonoheptanoic acid); APV (R-2-amino-5-phosphonopentanoate); CPPene (3-[(R)-2-carboxypiperazin-4-yl]-prop-2-enyl-1-phosphonic acid); (+)-(1S;2S)-1-(4-hydroxy-phenyl)-2-(4-hydroxy-4-phenylpiperidino)-1-pro-panol; (1S,2S)-1-(4-hydroxy-3-methoxyphenyl)-2-(4-hydroxy-4-phenylpiperi-dino)-1-propanol; (3R,4S)-3-(4-(4-fluorophenyl)-4-hydroxypiperidin-1-yl-)-chroman-4,7-diol; (1R*;2R*)-1-(4-hydroxy-3-methylphenyl)-2-(4-(4-fluoro-phenyl)-4-hydroxypiperidin-1-yl)-propan-1-ol-mesylate; or combinations thereof. In some embodiments, the NMDA receptor antagonist is not memantine.

### Methods of Treatment

Disclosed herein, in certain instances, are methods of treating bipolar disorder. In some embodiments, the bipolar disorder is childhood-onset bipolar disorder. In some embodiments, the bipolar disorder is childhood-onset bipolar disorder with Fear of Harm (FOH). In some embodiments, the bipolar-disorder is childhood-onset bipolar disorder which presents with at least one of the following symptoms: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of ketamine sufficient to alleviate symptoms associated with bipolar disorder, childhood-onset bipolar disorder, or childhood-onset bipolar disorder with Fear of Harm. In some embodiments, the ketamine is racemic. In some embodiments, the ketamine is (R)-ketamine. In some embodiments, the ketamine is (S)-ketamine.

Disclosed herein, in certain instances, are methods of treating at least one symptom associated with bipolar disorder. In some embodiments, the method comprises treating at least one symptom of childhood-onset bipolar disorder presenting with Fear of Harm (FOH). In some embodiments, the at least one symptom is: mood cycling (i.e., cycling between manic episodes, depressive episodes, and normal moods), obsessive fear of harm, severe aggression, territorial aggression, thermal dysregulation, night sweats, early and middle insomnia, arousal disorders of sleep (night mares, night-terrors, teeth grinding, bedwetting), rapid speech, germ contamination fears, hoarding, extreme separation anxiety, hallucinations, delusions, and sweet cravings. In some embodiments, the method comprises administering to a subject in need thereof at least one dose of ketamine sufficient to alleviate symptoms associated with bipolar disorder, childhood-onset bipolar disorder, or childhood-onset bipolar disorder with Fear of Harm. In some embodiments, the ketamine is racemic. In some embodiments, the ketamine is (R)-ketamine. In some embodiments, the ketamine is (S)-ketamine.

### Modes of Administration

In some embodiments, the ketamine is administered by any suitable method. In some embodiments, ketamine is administered orally, parenterally (e.g., intravenous, subcutaneous, intramuscular), intranasally, buccally, topically, rectally, or transdermally. In some embodiments, the ketamine is administered topically. In some embodiments, the ketamine is administered nasally. In some embodiments, the ketamine is administered parenterally. In some embodiments, the ketamine is administered by IV infusion.

In some embodiments, the ketamine is administered as an aerosol spray for nasal inhalation. In some embodiments, the ketamine is administered as a powder for nasal administration. In some embodiments, the ketamine is administered as a solution for nasal administration. In some embodiments, the ketamine is administered as a suspension for nasal administration.

In some embodiments, ketamine is administered intranasally as a single dose, such as a single daily dose. In some embodiments, ketamine is administered intranasally in multiple doses, such as multiple doses (e.g., 2, 3, 4, 5, 6, 7, 8, 9, 10, 15, 20, 25, and 50) doses of ketamine over a course of a day, days, weeks, or months. For example, the ketamine composition may be administered intranasally twice daily or four times weekly.

Any form of aerosolization known in the art, including but not limited to spray bottles, nebulization, atomization or pump aerosolization of a liquid formulation, and aerosolization of a dry powder formulation, is used in the practice of the invention. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

As used herein, the term “inhaler” refers both to devices for nasal and pulmonary administration of a drug, e.g., in solution, powder and the like. For example, a the term “inhaler” is intended to encompass a propellant driven inhaler, such as is used for to administer antihistamine for acute asthma attacks, and plastic spray bottles, such as are used to administer decongestants.

In some embodiments, the device for aerosolization is a metered dose inhaler. A metered dose inhaler provides a specific dosage when administered, rather than a variable dose depending on administration. Such a metered dose inhaler can be used with either a liquid or a dry powder aerosol formulation. Metered dose inhalers are well known in the art.

In some embodiments, the mass median dynamic diameter will be 5 micrometers or less in order to ensure that the drug particles reach the lung alveoli.

For nasal administration, a useful device is a small, hard bottle to which a metered dose sprayer is attached. In one embodiment, the metered dose is delivered by drawing the ketamine solution into a chamber of defined volume, which chamber has an aperture dimensioned to aerosolize and aerosol formulation by forming a spray when a liquid in the chamber is compressed. The chamber is compressed to administer the ketamine. In a specific embodiment, the chamber is a piston arrangement. Such devices are commercially available.

Alternatively, a plastic squeeze bottle with an aperture or opening dimensioned to aerosolize an aerosol formulation by forming a spray when squeezed. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

Often, the aerosolization of a liquid or a dry powder formulation for inhalation into the lung will require a propellent. The propellent may be any propellant generally used in the art. Specific nonlimiting examples of such useful propellants are a chlorofluorocarbon, a hydrofluorocarbon, a hydrochlorofluorocarbon, or a hydrocarbon, including trifluoromethane, dichlorodifluoromethane, dichlorotetrafluoroethanol, and 1,1,1,2-tetrafluoroethane, or combinations thereof.

### Dosage

In some embodiments, the method comprises administering a therapeutically effective amount of ketamine.

In some embodiments, the therapeutically-effective amount of ketamine is about 0.01 to about 1 mg/kg of body weight. In some embodiments, the therapeutically-effective amount of ketamine is about 0.05 to about 0.7 mg/kg of body weight.

The dose of ketamine that is administered generally will depend on the size of the subject being treated. In some embodiments, ketamine is administered at a dose of between about 0.1 mg/kg per day to about 3.0 mg/kg/day. The dose of ketamine may be from approximately 0.01 to approximately 1 mg/kg of body weight. In some embodiments, the dose of ketamine is approximately 0.05 to approximately 0.7 mg/kg of body weight. In other embodiments, the total dose of ketamine per nasal administration ranges from about 1 to about 250 mg. A dose of any integer between these two numbers is contemplated. Thus, for example, intranasal formulations respectively containing total intranasal doses of 1 mg, 2 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, and 250 mg are specifically contemplated.

In some embodiments, ketamine is administered at a dose of about 10% to about 20% of the amount used to induce anesthesia.

As bipolar disorder or mania is a chronic illness requiring maintenance treatment, it is expected that chronic administration of the intranasal formulation may be employed as necessary, ranging from daily to weekly, depending on response. For example, should the 50 mg intranasal dosage prove to be inadequate to treat bipolar disorder or mania effectively, increasing doses, e.g., approximately 100 mg, approximately 150 mg, approximately 200 mg, approximately 250 mg total ketamine may be administered intranasally.

The doses of ketamine used may be suitably modified to take into account the ketamine bioavailability so that the serum level of ketamine is less than, or on the order of 50 ng/ml.

Doses of ketamine far lower than those used in the treatment of other disorders are beneficial for subjects suffering from bipolar disorder or mania, in order to minimize the psychotomimetic side effects commonly associated with the use of the higher doses of ketamine. Also, it is suitable that the dose of ketamine administered is less than the dose generally administered for alleviation of pain.

The mild adverse effects of ketamine, e.g., dysphoria and/or hallucinations, sometimes called “ketamine dreams,” can occur upon administration of a dose of greater than 50 mg of ketamine, and usually require doses greater than 100 mg of ketamine of total dose intranasally. One advantage of the present invention is that nasal delivery of ketamine allows for control of the dose to a level effective for analgesia, but below the level that results in such dreams.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. An agent disclosed herein exhibiting high therapeutic indices is preferred. The data obtained from cell culture assays and animal studies are optionally used in formulating a range of dosage for use in human. The dosage of such an agent disclosed herein lies preferably within a range of circulating concentrations that include the ED50 with minimal toxicity. The dosage optionally varies within this range depending upon the dosage form employed and the route of administration utilized.

In the case wherein the individual's condition does not improve, upon the doctor's discretion the administration of an agent disclosed herein is optionally administered chronically, that is, for an extended period of time, including throughout the duration of the individual's life in order to ameliorate or otherwise control or limit the symptoms of the individual's disease or condition.

In the case wherein the individual's status does improve, upon the doctor's discretion the administration of an agent disclosed herein is optionally given continuously; alternatively, the dose of drug being administered is temporarily reduced or temporarily suspended for a length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the individual's conditions has occurred, a maintenance dose is administered if necessary. Subsequently, the dosage or the frequency of administration, or both, is reduced, as a function of the symptoms, to a level at which the improved disease, disorder or condition is retained. In some embodiments, individuals require intermittent treatment on a long-term basis upon any recurrence of symptoms.

### Pharmaceutical Compositions

Disclosed herein, in some embodiments, is a pharmaceutical composition for treating a bipolar disorder, condition, or symptom comprising a therapeutically-effective amount of ketamine. In some embodiments, the bipolar disorder is childhood-onset bipolar disorder. In some embodiments, the bipolar disorder is childhood-onset bipolar disorder with FOH.

Pharmaceutical compositions herein are formulated using one or more physiologically acceptable carriers including excipients and auxiliaries which facilitate processing of the agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, a composition disclosed herein is formulated as a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol.

In some embodiments, a composition disclosed herein is formulated as a microencapsulated tablet. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a MIF receptor inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In some embodiments, a composition disclosed herein is formulated as a liquid (e.g., dispersions, emulsions, solutions, elixirs, gels, and syrups). See, e.g., Singh et al., Encyclopedia of Pharmaceutical Technology, 2nd Ed., pp. 754-757 (2002).

In some embodiments, a composition disclosed herein is formulated for topical administrations (e.g., as a solution, suspension, lotion, gel, paste, medicated stick, balm, cream or ointment). Such pharmaceutical compositions optionally contain solubilizers, stabilizers, tonicity enhancing agents, buffers and preservatives.

In some embodiments, a composition disclosed herein is formulated for intranasal administration. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the pharmaceutical compositions disclosed herein are optionally in a form of an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix and a suitable powder base such as lactose or starch.

In some embodiments, a composition disclosed herein is formulated for buccal administration. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period. Buccal drug delivery avoids the disadvantages encountered with oral drug administration, e.g., slow absorption, degradation of the agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which is obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

In some embodiments, a composition disclosed herein is formulated for transdermal administration. Transdermal formulations include at least three components: (1) an agent; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery is optionally accomplished by means of iontophoretic patches and the like. Additionally, transdermal patches provide controlled delivery. The rate of absorption is optionally slowed by using rate-controlling membranes or by trapping an agent within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing an agent optionally with carriers, optionally a rate controlling barrier to deliver a an agent to the skin of the host at a controlled and predetermined rate over a prolonged period of time, and means to secure the device to the skin.

In some embodiments, a composition disclosed herein is formulated for parenteral administration. Formulations suitable for parenteral administration include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

Parenteral formulations are optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent in water soluble form. Additionally, suspensions are optionally prepared as appropriate oily injection suspensions.

In some embodiments, a composition disclosed herein is formulated for rectal administration (e.g., as an enema, rectal gel, rectal foam, rectal aerosol, suppository, jelly suppository, or retention enema), containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In some embodiments, the pharmaceutical composition described herein is in unit dosage forms suitable for single administration of precise dosages. In unit dosage form, the formulation is divided into unit doses containing appropriate quantities of an agent disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

In some embodiments, a pharmaceutical composition comprising ketamine further comprises a pharmaceutically acceptable diluent(s), excipient(s), or carrier(s). In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, buffers, antioxidants, and/or flavoring agents. In addition, the pharmaceutical compositions also contain other therapeutically valuable substances.

### Carriers

In some embodiments, a composition comprising ketamine further comprises a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half life for clearance. Such macromolecules include but are not limited to Soya lecithin, oleic acid and sorbitan trioleate, with sorbitan trioleate preferred.

### Buffers

In some embodiments, a composition comprising ketamine further comprises a buffer. Buffers may be added to maintain the pH of the formulation to between about 3 to about 8 (e.g., about 3, about 3.5, about 4, about 4.5, about 5, about 5.5, about 6, about 6.5, about 6.8, about 7, about 7.2, about 7.3, about 7.4, about 7.5, about 7.6). According to some embodiments, the pH of the formulation may be between about 3 and about 6, between about 3 and about 5.5, between about 3 and about 5.2, between about 3 and about 4.5, between about 3 and about 4, between about 4 and about 5.5, between about 4.5 and about 5.5, between about 4 and about 6, between about 4 and about 7, between about 3 and about 7, between about 4 and about 7.5, between about 5 and about 7.5, between about 5 and about 6.5, between about 5 and about 8, between about 6 and about 8, or between about 6.5 and about 7.5.

Any pharmaceutically acceptable buffer may be used in the present formulations/compositions. Preferably, the buffer is present in a concentration of from about 1 mM to about 100 mM (e.g., about 5 mM, about 10 mM, about 15 mM, about 20 mM, about 25 mM, about 30 mM, about 35 mM, about 40 mM, about 50 mM, about 55 mM, about 60 mM, about 65 mM, about 70 mM, about 75 mM, about 80 mM, about 85 mM, about 90 mM, or about 95 mM).

The buffer to be included in the formulations may include, but is not limited to, the following: acetate (e.g., sodium acetate), sodium carbonate, citrate (e.g., sodium citrate), tartrate, glycylglycine, histidine, glycine, lysine, arginin, sodium dihydrogen phosphate, disodium hydrogen phosphate, sodium phosphate, and tris(hydroxymethyl)-aminomethan, or mixtures thereof. Each one of these specific buffers constitutes an alternative embodiment of the invention. In a preferred embodiment of the invention the buffer is glycylglycine, sodium dihydrogen phosphate, disodium hydrogen phosphate, sodium phosphate or mixtures thereof.

### Bulking Agents

In some embodiments, a composition comprising ketamine further comprises a bulking agent. Without limitation, the bulking agent may be one or more of the following: lactose, sorbitol, sucrose, and mannitol.

### Dispersants

In some embodiments, a composition comprising ketamine further comprises a dispersant. Despersants include, but are not limited to, to surfactants such as polyoxyethylene fatty acid esters and alcohols, and polyoxyethylene sorbitan fatty acid esters. Amounts of surfactants used will vary, being generally within the range or 0.001 and 4% by weight of the formulation. Suitable surfactants are selected on the basis of desired properties, depending on the specific formulation, concentration of ketamine, diluent or form of ketamine.

### Penetration Enhancers

In some embodiments, a composition comprising ketamine further comprises a penetration enhancer. Penetration enhancer include, but not limited to, a bile salt, fatty acid, surfactant or alcohol. In specific embodiments, the permeation enhancer can be sodium cholate, sodium dodecyl sulphate, sodium deoxycholate, taurodeoxycholate, sodium glycocholate, dimethylsulfoxide or ethanol.

### Preservatives

In some embodiments, a composition comprising ketamine further comprises a preservative. Where a pharmaceutically acceptable preservative is to be included in the formulations of the invention, the preservative is selected from the group consisting of phenol, m-cresol, benzalkonium chloride, chloroethanol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, or mixtures thereof. Each one of these specific preservatives constitutes an alternative embodiment of the invention. In a preferred embodiment of the invention the preservative is benzyl alcohol, a phenol, or m-cresol.

In a further embodiment of the invention the preservative is present in a concentration from about 0.1 mg/ml to about 50 mg/ml, more preferably in a concentration from about 0.1 mg/ml to about 25 mg/ml, and most preferably in a concentration from about 0.1 mg/ml to about 10 mg/ml.

### Sterility

In some embodiments, a composition disclosed herein is sterile. It is preferred that the compositions disclosed herein be free of pathogenic organisms. Any suitable process of sterilization is used with a composition disclosed herein. In one embodiment, a composition disclosed herein is produced under sterile conditions. In alternative embodiment, a composition disclosed herein is sterile filtered and filled in vials, including unit dose vials providing sterile unit dose formulations. Each unit dose vial may be sterile and is suitably administered without contaminating other vials or the next dose. In alternative embodiment, a composition disclosed herein is sterilized by heat (e.g., steam) or radiations (e.g., gamma radiation). Compositions are prepared and handled under sterile conditions, or are sterilized before or after packaging.

### EXAMPLES

### Example 1

### Linking of Fear of Harm (FOH) Phenotype with Childhood-Onset Bipolar Disorder

### Methods

A sample was comprised of children with community diagnoses of bipolar disorder or at risk for the illness based on enriched family history with multiple first degree relatives diagnosed with bipolar disorder (BPD) (N=5335). Included were all subjects who had N40 positively endorsed Childhood Bipolar Questionaire (CBQ) symptom items at frequencies of very often, almost always, and always. This group was divided randomly into two groups, the exploratory group (N=2668) and the study group (N=2666). The exploratory group was used for exploratory data analysis and the development of hypotheses. A study group was used to test these hypotheses using a new and uncontaminated set of data. All results reported here are derived from the latter group. In subsequent analyses, a subset of the study group sample was examined for differences in age of onset of first psychiatric symptoms, course of illness and measures of symptom severity. These groups were compared using the chi-square procedure for categorical data and the Analysis of Variance (ANOVA) with Scheffe pair wise tests for continuous variables. The Child Bipolar Questionnaire V.2.0, the Yale-Brown Obsessive Compulsive Scale (YBOCS) and the Overt Aggression Scale (OAS) were the principal instruments used to obtain diagnostic information for this study.

To assess the relationship between membership in an FOH group and symptoms of mood dysregulation and psychiatric disorders, the CBQ was administered to all subjects (N=1726). The CBQ is a 65 item, self-administered, parent report measure originally developed to establish initial eligibility for clinical and genetic studies of PBD (Papolos et al., J. Affect. Disord. 95: 149-158 (2006), incorporated herein by reference in its entirety). It was constructed based on the model proposed by Depue et al., J. Abnorm. Psychol. 90: 381-437 (1981), incorporated herein by reference in its entirety, who, with the development and validation of the General Behavior Inventory (GBI), derived a dimensional approach for the definition of BPD in adults. Behaviors and symptoms are measured on 1-4 Likert scale. A rapid screening instrument with a Core Index subscale of key symptom dimensions frequently reported in PBD, the CBQ includes scoring algorithms for DSM-IV BD, with and without attention deficit/hyperactivity disorder (ADHD). Test/retest data showed excellent reliability for both the CBQ total score (r=0.82) and the Core Index subscale (r=0.86). CBQ screening algorithms were performed with a specificity of 97% and a sensitivity of 76% in classifying subjects with Kiddie Schedule for Affective Disorders and Schizophrenia (K-SADS P/L) diagnosis of BD vs. no BD (Papolos et al., 2006). The Core Index subscale had excellent agreement with K-SADS P/L diagnosis (k=0.84) in classifying BD, ADHD-only, and no diagnosis and demonstrated 100% sensitivity and 86% specificity in classifying BD vs. no BD. Consistent with a previous examination of the FOH symptom dimension (Papolos et al., J. Affect. Disord. 89: 99-105 (2005a), incorporated herein by reference in its entirety), a Yale-Brown Obsessive Compulsive Scale (YBOCS) measure was used that consisted of a count of six aggressive obsessions rated by the parent as occurring at a frequency of “often” or “very often” or “almost constantly”: fear might harm self; fear might harm others; fear harm might come to self; fear harm will come to others (may be because of something child did or did not do); fear will act on unwanted impulses (e.g., to stab a family member); and fear will be responsible for something else terrible happening (e.g., fire, burglary, flood). The FOH index was calculated by summing six YBOCS items that had scored greater or equal to 3 and two items from OAS that had scored greater or equal to 2. The items from the OAS are: mutilates self, causes deep cuts, bites that bleed internal injury, fracture, loss of consciousness, loss of teeth and attacks others, causing severe physical injury.

Consistent with a previous examination of the FOH symptom dimension (Papolos et al., 2005a and Papolos et al. J. Affect. Disord. 86: 267-275 (2005b), incorporated herein by reference in its entirety) YBOCS items that had scored greater or equal to 3 and two items from OAS that scored greater or equal to 2 defined the phenotype. A principal component factor analysis with Varimax rotation was used to determine what other traits are associated with the FOH trait by examining the independent factors derived from the CBQ. To determine the nature and extent to which each of these factors were associated with the FOH trait, a total score for each factor was calculated by summing all items for each factor and the factors were named based on item content. Cronbach's alpha was also calculated per factor. These factors were used in a multiple regression model to predict the Fear of Harm Index using a stepwise method. Some questions were not applicable to all subjects, resulting in different sample size per variable. The SPSS version 15 was used for all these analyses.

### Results

Of the 2666 subjects, 1729 were found to have FOH data. When the distribution of the FOH index in this sample was examined, it was found that a full third of the group had no FOH (X²=169.14, df=1, pb.001). The total group of 1729 children was, therefore, divided into three groups. A group with no FOH symptoms (NoFOH), values of 0 positively endorsed items (NoFOH: 0±0, N=621, 36%), and subjects with values from 1 through 7 (LowFOH: 4.5±2N=555, 32%) were designated as the low FOH group. The high FOH group included subjects with values greater than or equal to seven (HighFOH: 14.1±5, N=553, 32%).

Although there were no significant differences between rates of males and females on the Fear of Harm Index (female: 5.7±6, male: 6.2±7, f=2.1, df=1,1640, p=0.148), there were significantly more male subjects in the LowFOH group (NoFOH: 35%, LowFOH: 45%, and HighFOH: 34%, X²=6.41, df=2, p=0.041). There was no significant age difference among groups (NoFOH=10.0±4, N=585; LowFOH=10.2±4, N=528; HighFOH=10.4±4; f=1.7; df=2,1636; p=0.182). However, there were significantly more ADHD subjects in the NoFOH group compared to HighFOH (NoFOH=19%, Low-FOH=16%, HighFOH=11%, X²=7.9, df=1, p=0.005).

Despite the fact that the three groups did not differ on the number of subjects diagnosed with bipolar disorder (NoFOH=83%, LowFOH=86%, HighFOH=86%, X²=1.13, df=2, p=0.57), or major depressive disorder (NoFOH=4%, LowFOH=2%, HighFOH=2%, X²=2.69, df=2, p=0.26), using CBQ item scores we found that there was a significantly greater frequency of manic (NoFOH=5.0±2, LowFOH=5.7±1, HighFOH=5.6±2; f=79.43; df=2,1726; pb.0001) and depressive symptoms (NoFOH=3.9±2, LowFOH=4.6±2, HighFOH=4.9±2; f=60.53; df=2,1726; pb.0001) in the high FOH group when compared to the low or no FOH groups. Pairwise tests indicate that all groups are significantly different from each other on these variables. These differences are also evident when the dimensions were dichotomized (Table 1). The HighFOH group has a significantly greater number of subjects with five or more manic/hypomanic symptoms, 91%, compared to the LowFOHgroup of 83% and NoFOHgroup of 69% of subjects (X²=93.8, df=2, pb.000). All pair wise comparisons were also significant. The differences persisted when analyzed for depressive symptoms; 84% of the HighFOH group exhibited four or more symptoms of depression in comparison to 78% of the LowFOH and 62% of NoFOH groups (X²=76.4, df=2, pb.0001). All pair wise comparisons were also significant. Similar results were found when groups were compared separately for male and female subjects (Table 1).

TABLE 1

| | No FOH | Low FOH | High FOH | X² (p < .001) |
|---|---|---|---|---|
| Manic symptoms | 69% (426) | 83% (459) | 91% (502) | 93.8 |
| greater or | | | | |
| equal to 5 | | | | |
| Depressive | 62% (387) | 78% (431) | 84% (464) | 76.4 |
| symptoms greater | | | | |
| or equal to 4 | | | | |

Course of illness data were available for 967 children. Within this subgroup the same criteria was applied for FOH status. Similar to the larger pool of children, this smaller group contained about a third of children who endorsed 0 items of FOH(N=334, 35%), a third endorsed 1 through 7 items (N=322, 33%) and a third endorsed more than 7 items (N=311, 32%). The similarity of the distribution of FOH in each group raises one's confidence that this smaller subset of children is a representative sample of the larger sample.

The three groups endorsed CBQ items significantly differently from each other (f=137.69; df=2,981; pb.001). The NoFOH group positively endorsed 37.9±11 items, LowFOH 45.8±8 items and the HighFOH group positively endorsed 49.6±8 items. These differences were similar to the larger group. The groups were not significantly different in age (NoFOH=9.7±4, LowFOH=9.9±4, HighFOH=10.3±4; f=2.10; df=2,896; p=0.122). The groups had a similar distribution of male subjects (NoFOH: 33%, LowFOH: 30%, and HighFOH: 36%, X2=5.11, df=2, p=0.077).

These groups had a similar age of onset of first reported psychiatric symptoms, age of initial psychiatric evaluation, age of initial diagnosis and age at first psychiatric hospitalization. However, they were significantly different on the number of hospitalizations (Table 2).

TABLE 2

| | No FOH | Low FOH | High FOH | f | p < .01 |
|---|---|---|---|---|---|
| Age of 1^{st} symptom (years) | 2.7 ± 2 (334) | 2.6 ± 3 (322) | 2.5 ± 2 (311) | 1.12 | .326 |
| Age of initial psychiatric evaluation (years) | 6.0 ± 3 (316) | 6.0 ± 3 (312) | 6.0 ± 3 (300) | .037 | .963 |
| Age of initial diagnosis (years) | 6.3 ± 3 (306) | 6.5 ± 5 (313) | 6.3 ± 4 (302) | .365 | .694 |
| Age of 1^{st} psychiatric hospitalization (years) | 9.7 ± 4 (78) | 9.6 ± 4 (114) | 9.4 ± 4 (164) | .337 | .713 |
| Number of hospitalizations | 1.5 ± 1 (86) | 1.8 ± 2 (118) | 2.4 ± 2 (172) | 6.31 | .002 |

The NoFOH group has a significantly fewer number of hospitalization than the other two groups.

On measures of severity of illness presented in Table 3, there were significant differences found among the FOH groups on the severity of illness variables; Ever Hospitalized, Held Back a Grade, and Suspended from School.

TABLE 3

| | Yes | No FOH | Low FOH | High FOH | X² | p value |
|---|---|---|---|---|---|---|
| Ever | 352 | 22 | 34 | 52 | 63.7 | .001 |
| hospitalized | | | | | | |
| (984) | | | | | | |
| Home | 40 | 5.4 | 4.3 | 2.2 | 4.7 | .094 |
| schooled | | | | | | |
| (984) | | | | | | |
| Held back a | 171 | 15 | 20 | 24 | 8.5 | .014 |
| grade (880) | | | | | | |
| Ever | 366 | 36 | 38 | 48 | 9.9 | .007 |
| suspended | | | | | | |
| from school | | | | | | |
| (905) | | | | | | |
| Involved | 110 | 91 | 89 | 86 | 5.1 | .079 |
| with the | | | | | | |
| juvenile | | | | | | |
| justice | | | | | | |
| system | | | | | | |
| (984) | | | | | | |

However, the groups were not significantly different on home schooling and their involvement with the juvenile justice system. All groups were significantly different from each other on ever hospitalized with HighFOH has the largest percentage of subjects (52%). Significantly more subjects from the HighFOH group were also held back a grade compared to NoFOH(X²=8.49, df=1, p=0.004) and significantly more subjects from HighFOH and LowFOH were suspended from school than NoFOH (X²=8.48, df=1, p=0.004; X²=6.24, df=1, p=0.012). There was a strong trend between held back a grade and suspended from school. 47% of subjects who were held back a grade were also suspended from school (X²=2.75, df=1, p=0.098). 14% of subjects from HighFOH groups were suspended from school and held back a grade compared to 7% from NoFOH (X²=7.39, df=1, p=0.007) and 6% subjects from LowFOH groups (X²=11.30, df=1, p=0.001).

Using all of the children in the study, a principal component factor analysis was used to identify a set of independent dimensions associated with the FOH trait of children (N=1729; NoFOH=621, LowFOH=555, High-FOH=553). The factor analysis yielded thirteen factors with eigen values greater than 1.0 that explained a total of 61% of variance. By combining 3 of the factors with the lowest Cronbach's alpha with other factors to which they also contributed, the 13 factors were reduced to 10. These ten factors, their CBQ items, Eigenvalues, percentage of variance, and the Cronbach's alphas are listed in Table 4.

TABLE 4

| Factor | CBQ items | Eigenvalues | % Variance | α |
|---|---|---|---|---|
| Factor 1: Territorial | 46) is willful and refuses to be subordinated by others | 16.56 | 25.5 | .91 |
| Aggression | 47) argues with adults | | | |
| | 49) defies or refuses to comply with rules | | | |
| | 51) is easily angered in response to limit setting | | | |
| | 48) is bossy towards others | | | |
| | 45) relentlessly pursues own needs and is demanding | | | |
| | of others | | | |
| | 50) blames others for his/her mistakes | | | |
| | 53) has protracted, explosive temper tantrums | | | |
| | 55) displays aggressive behavior towards others | | | |
| | 32) has irritable mood states | | | |
| | 52) lies to avoid consequences of his/her actions | | | |
| | 44) is intolerant of delays | | | |
| | 54) has difficulty maintaining friendships | | | |
| Factor 2: Attention/Executive | 17) has difficulty organizing tasks | 3.71 | 5.7 | .87 |
| function | 13) demonstrates inability to concentrate at school | | | |
| | 12) is easily distracted during repetitive chores and | | | |
| | lessons | | | |
| | 14) attempts to avoid homework assignments | | | |
| | 16) has poor handwriting | | | |
| | 11) is easily distracted by extraneous stimuli | | | |
| | 19) has difficulty estimating time | | | |
| | 15) able to focus intently on subjects of interest and | | | |
| | yet at times is easily distractible | | | |
| | 20) has auditory processing or short-term memory deficit | | | |
| | 18) has difficulty making transitions | | | |
| Factor 3: Mania | 25) has periods of high, frenetic energy and motor activation | 3.24 | 4.9 | .87 |
| | 28) has periods of excessive and rapid speech | | | |
| | 26) has many ideas at once | | | |
| | 33) has elated or silly, goofy, giddy mood states | | | |
| | 24) is easily excitable | | | |
| | 27) interrupts or intrudes on others | | | |
| | [symbol] 4) is hyperactive and easily excited in the PM | | | |
| | 31) displays abrupt, rapid mood swings | | | |
| | 43) fidgets with hands or feet | | | |
| | 65) is very intuitive and/or very creative | | | |
| | 30) tells tall tales; embellishes or exaggerates | | | |
| | 29) has exaggerated ideas about self or abilities | | | |
| Factor 4: Harm to Self/Others | 59) makes clear threats of violence to others or self | 2.93 | | .83 |
| | 58) makes moderate threats to others or self | | | |
| | 60) has made clear threats of suicide | | | |
| | 57) curses viciously, uses foul language in anger | | | |
| | 56) has destroyed property intentionally | | | |
| | 61) is fascinated with gore, blood, or violent imagery | | | |
| Factor 5: Self-esteem | 41) feels easily criticized and/or rejected | 2.40 | | .84 |
| | 42) feels easily humiliated or shamed | | | |
| | 40) experiences periods of self doubt and poor self-esteem | | | |
| | 37) complains of being bored | | | |
| | 38) has periods of low energy and/or withdraws or | | | |
| | isolates self | | | |
| | 39) has decreased initiative | | | |
| Factor 6: Sleep | [symbol] 6) has difficulty getting to sleep | 1.93 | | .74 |
| | [symbol] 5) has difficulty settling at night | | | |
| | [symbol] 7) sleeps fitfully and/or awakens in the middle of | | | |
| | the night | | | |
| | [symbol] 3) has difficulty arising in the AM | | | |
| Factor 7: Sensory | 21) is extremely sensitive to textures of clothes, labels, | 1.78 | | .71 |
| | and tightness of fit of socks or shoes | | | |
| | 22) exhibits extreme sensitivity to sound and noise | | | |
| | 23) complains of body temperature extremes or feeling | | | |
| | hot despite neutral ambient temperature | | | |
| Factor 8: Hypersexuality | 34) displays precocious sexual curiosity | 1.50 | | .74 |
| | 35) exhibits inappropriate sexual behaviors, e.g. openly | | | |
| | touches self or others' private parts | | | |
| | 36) takes excessive risks | | | |
| Factor 9: Psychosis, | 09) wets bed | 1.23 | | .59 |
| Parasomnias, Sweet | 08) has night terrors and/or nightmares | | | |
| Cravings, | 63) hoards or avidly seeks to collect objects or food | | | |
| and Obsessions | 62) has acknowledged experiencing auditory and/or | | | |
| | visual hallucinations | | | |
| | 10) craves sweet-tasting foods | | | |
| | 64) has concern with dirt, germs, or contamination | | | |
| Factor 10: Anxiety | 01) displays excessive distress when separated from | 1.03 | | .66 |
| | family | | | |
| | 02) exhibits excessive anxiety or worry | | | |

α: Cronbach's alpha. [symbol]
indicates data missing or illegible when filed

Descriptive information for each CBQ factor for the three FOH groups are presented in Table 5.

TABLE 5

| | | | | Factor group |
|---|---|---|---|---|
| | No FOH | Low FOH | High FOH | mean |
| Territorial | 39.8 ± 9 | 43.5 ± 7 | 46.5 ± 5 | 43.1 ± 8 |
| aggression | | | | |
| Attention/executive | 30.9 ± 9 | 33.1 ± 5 | 34.5 ± 5 | 32.8 ± 6 |
| function | | | | |
| Mania | 36.1 ± 7 | 39.0 ± 6 | 41.3 ± 5 | 38.7 ± 7 |
| Harm to self/ | 11.9 ± 4 | 14.5 ± 4 | 17.6 ± 4 | 14.5 ± 5 |
| others | | | | |
| Self-esteem | 17.3 ± 4 | 18.9 ± 4 | 20.1 ± 4 | 18.7 ± 4 |
| Sleep | 11.5 ± 3 | 12.5 ± 3 | 13.0 ± 3 | 12.3 ± 3 |
| Sensory | 7.4 ± 3 | 8.1 ± 3 | 8.8 ± 3 | 8.1 ± 3 |
| Hypersexuality | 5.7 ± 2 | 6.5 ± 3 | 7.3 ± 3 | 6.5 ± 3 |
| Psychosis/ | 11.5 ± 3 | 13.2 ± 3 | 14.6 ± 4 | 13.0 ± 4 |
| parasomnias/ | | | | |
| sweet cravings/ | | | | |
| obsessions | | | | |
| Anxiety | 4.7 ± 2 | 5.5 ± 2 | 5.9 ± 2 | 5.3 ± 2 |

The mean number of CBQ items endorsed by the three FOH groups was significantly different from each other. The NoFOH group positively endorsed 37.9±11 items (out of 65 items), LowFOH 45.05±9 items and the HighFOH group positively endorsed 49.99±8 items (f=243.27; df=2,1726; pb.001). Subjects who scored either HighFOH or LowFOH were found to have more severe symptoms on all of these CBQ factors than children without the FOH trait.

We performed a logistic regression analysis of ten independent CBQ factors in order to determine which individual set of behavioral traits is the best predictor of FOH. A four factor model, (X²=1601, df=4, pb.001) including, Territorial Aggression, Harm to Self and Others, Selfesteem, and Psychosis/Parasomnias/Sweet Craving/Obsessions (PPSO) correctly predicted the FOH group with 96% accuracy.

Thus, subjects that were diagnosed with bipolar disorder using the CBQ who were positive for FOH had more hospitalizations, were more likely to be held back a grade, and more likely to be suspended from school than subjects with. Thus, subjects diagnosed with childhood-onset bipolar disorder with FOH may have more severe symptoms than subjects without FOH.

### Example 2

### Clinical Trail Evaluating Ketamine for Treatment of Childhood-Onset Bipolar Disorder with FOH

### Objectives

A four-arm clinical trial to evaluate the potential for efficacy of ketamine in the treatment of PBD/FOH. This will be a double-blind randomized discontinuation placebo-controlled study conducted to assess the efficacy and safety of the intranasal administration of the NMDA-antagonist ketamine, in the treatment of bipolar I and bipolar II disorders with FOH phenotype.

Ho: There is no difference in the changes of PBD/FOH symptom manifestation from baseline while treated with ketamine.

HA: There will be fewer and/or less severe PBD/FOH symptoms from baseline while being treated with an optimal dose of ketamine in both intranasal and oral form.

Secondary outcome measures of interest include: changes in P/D ratio of body temperature at sleep-onset and sleep-rage episodes, disturbances in sleep-onset and offset, sleep intertia, arousal disorders of sleep, restlessness, distractibility, impulsivity psychosis, complaints of boredom,

### Trial Design

Patients will have to meet the following inclusion criteria to be considered for participation in this trial: 1) at least 6 years of age 2) confirmed pre-pubertal onset bipolar disorder and 3) have failed to respond to prior treatment (needs to be more concretely defined) 4) meet symptomatic threshold (define)

Patients will be excluded from this trial based on the following criteria: 1) have any contraindication to the use of ketamine 2) endocrine or neurological illness, previous history of closed head injury loss of consciousness, dissociative responses to anesthesia.

Patients who meet the inclusion criteria without meeting the exclusion criteria and their parent/guardian will be invited to speak with a research assistant to discuss the procedures, benefits and potential risks involved with the study. The research assistant will emphasize the voluntary nature of the trial, and that participation can be terminated at the patient's will at any time for any reason.

Ascertainment of subjects will occur through the Juvenile Bipolar Research Foundation (JBRF) website. JBRF is a non-profit foundation dedicated to the support of research for the study of early-onset bipolar disorder. Psychiatrists in the New York metropolitan area Board certified research psychiatrists and APNs will conduct an initial screening of the candidates using the CBQ, OAS, MRS, and the Y-BOCs.

### Organization of Study Arms

Subjects will be randomized into one of two study arms. The first arm will take ketamine daily for three weeks intranasally, followed by a 12 day interruption of ketamine and ending with three weeks of orally administered ketamine extended release twice daily. The second arm will take ketamine for three weeks intranasally followed by 12 day interruption of ketamine, followed by one week of orally administered ketamine twice daily, ending with placebo until relapse. Following relapse, the subject will be resume oral ketamine with time to relapse noted.

The length of the active/placebo interruption may vary. The final phase of oral ketamine/placebo will commence at the time of symptom return. The “return of symptoms” will be strictly defined to take into account the individual's original constellation and severity of pre-trial symptoms, and the degree of relief from ketamine/placebo. The end of the placebo arm interruption may be more strictly defined with a minimum time frame as we do not expect any relief which would preclude a “return of symptoms.”

At the conclusion of the individual observation period, non-responders to placebo will be given the opportunity to enter treatment with ketamine. Patients will be randomized to a treatment arm after the screen and consenting and assenting process. The patient, parent, and nurse practitioner will be blinded from the treatment assignment. Following randomization, the patient will begin receiving ketamine administered either intranasally or orally.

Dosing will be determined on an individual basis, optimizing for inter-individual variability such as weight. Intranasal dosing will begin with one spray to one nostril at a concentration of 100 mg/mL increasing at a rate of one spray/qd until side effects intervene and/or the subject experiences significant reductions in BPD symptoms (to be defined). Oral dosing will begin with xmg/kg administered 2× day with increments of increasing at a rate of 5 mg/day with the same stopping rules as with the intranasal spray. In the second phase of medication, oral administration will continue at the previously established dose or oral equivalent.

### Data Collection

A structured diagnostic interview the Kidde-SADS (K-SADS) will be administered to a parent to confirm eligibility for this clinical trial. Two board-certified psychiatrists will diagnose those who have met all other inclusion criteria. The K-SADS-PL is a semi-structured diagnostic interview designed to assess the presence of present and lifetime psychiatric disorders in children and adolescents using DSM criteria. The K-SADS-PL has high content validity and provides global and diagnosis-specific impairment ratings. (Kaufman and Schweder, 2003 The Schedule for Affective Disorders and Schizophrenia for School Age Children: Present and Lifetime Version (K-SADS-PL).

The CBQ and temperature readings taken for one day will be used to categorize the subjects into the non-FOH and FOH groups. Subjects who exhibit thermoregulation dysfunction and have a FOH index score equal to or greater ______ than will be categorized as FOH. Subjects who do not meet both criteria will be categorized as non-FOH.

After the patient and family have provided consent and assent, but prior to the introduction of medication, preliminary data will be obtained. The nurse practitioner will evaluate the subject using a profile of neuropsychological tests that will measure neuropsychological function and attention. One of the tests will be the Connor's continuous performance test, a psychometric instrument used to assess attention deficits and neurological function. The CPT is a brief 14-minute computer activity that aids in the identification of attention disorders in persons 6 years and older (Conners 1985; Conners 2000) (cannot find any reliability/validity studies). [Conners, C. K. & MHS Staff. (Eds.) (2000) Conners' Continuous Performance Test II: Computer Program for Windows Technical Guide and Software Manual. North Tonwanda, N.Y.: Mutli-Health Systems.]

These data will include an interview with parents, interview with the child, and both parent and a nurse practitioner report of symptoms as demonstrated by the Mania Rating scale (MRS), the Overt Aggression scale (OAS), the Yale Brown Obsessive-compulsive scale (YBOCs) and the CBQ.

The Mania Rating Scale is a psychometric tool used to assess the severity of mania. The MRS has high overall reliability of 0.93 and strong validity as compared with of an independent global rating, the Petterson Scale, and the Beigel Scale (Young et al. 1978).

The Overt Aggression Scale is designed to measure aggressive behaviors in four categories: verbal aggression, physical aggression against objects, physical aggression against onself and physical aggression against others. The OAS has high reliability with an overall intraclass coefficient of 0.87 (Yudofsky et al. 1986).

The Y-BOCS is a clinician-administered semi-structured interview that serves a gold-standard for rating obsessive-compulsive symptoms. It shows high inter-rater reliability with an r=0.98 (Goodman et al. 1989).

Subjects will also have a physical examination, blood tests, weight measure, and electrocardiogram (ECG) at the beginning and end of the study.

After the commencement of medication, evaluation of subjects will occur at two levels, daily assessments and more in-depth assessments occurring at the end of each of the three phases (medication, interruption and medication). During the intranasal phase, the nurse practitioner will evaluate the subject using the dissociative states questionnaire for the two hour period following medication administration daily. At two hours, the nurse practitioner will interview the child and ask for the parent's observation in the changes child's symptoms during the intranasal period. The Mania Rating scale, the CBQ, and questions from the YBOCS (need to specify which questions) will be administered to the parents by the nurse practitioner twice a week during the entire study. Observation of fearful behaviors, body temperature changes, anxiety levels, and attentional processes will be observed through specific questions. These assessments will include a behavioral inventory (will define later) and physiological measures including temperature readings (core and peripheral). The in-depth psychological assessments would occur three times during the trial and include the completion of the CBQ and the Jeannie and Jeffrey Interview.

Oximetry (measurement of blood oxygen), pulse, and blood pressure are measured continuously for 1 hour before and 4 hours after each ketamine or placebo dose to monitor safety. Interviews and rating scales: Patients complete a series of psychiatric rating scales to assess the effects of the study drug on mood and thinking. The rating scales are repeated up to 18 times during the study, with each time taking about 15 to 20 minutes.

### Safety Consideration

Given that this study is proposed for a pediatric population, additional safeguards will be taken to ensure their protection. During the data collection, safety measures will be taken from the child, parent and overseeing nurse practitioner.

The main side effects of concern that may occur with ketamine use include dissociation, hallucinations, vomiting, depressed respiration, and addiction. The Dissociative scale will be used after each intra nasal administration and daily during oral administration to assess dissociation until 7 days after stabilization of dose symptom relief (do we expect these to either present themselves before this time or are they only acute problems?).

Other less serious adverse effects that may include a bitter taste, and a sensation of burning in the pharynx which have been seen after acute intranasal use of ketamine in children.

### Example 3

### Ketamine Treatment of Childhood-Onset Bipolar Disorder With FOH

Subject is a 3 yrs and 5 months old girl referred by her parents for psychiatric care to address her intense and often violent rages.

### Reason for Referral

Subject has had episodic rages that have escalated in frequency and intensity since her adoption at 18 months. She has always experienced extreme separation anxiety but her aggression has shifted from herself toward her parents since starting school. The abuse towards her parents has included hitting, kicking, biting, and propelling objects. Parents came for consultation for fear that Subject will become more dangerous as she ages.

### Birth and Development

Subject was delivered at 9 months via caesarian section with reports of a “near-miscarriage.” She remained at the hospital for 2 months before being placed in an orphanage. Subject was bottle-fed from birth, walked at 1 year, and bladder-trained by 18 months. She made sounds before 18 months, but did not begin forming words until 18 months. Subject was diagnosed with speech delay involving oral motor articulation and after therapy, she was diagnosed with auditory processing disorder shortly before her 3rd birthday.

### Medical History

Subject has recurrent ear infections from before 18 months with scarring of both ears. She had bilateral pneumonia, several bronchial infections and chicken pox prior to 18 months. At 2 years old, Subject had myringotomy tubes inserted bilaterally. Her adenoids were removed at age 3. Versid was met with extreme hyperactivity instead of relaxation. A similar response occurred with Tylenol and codeine. From 18 months to 3 years, Subject resisted sleep with rages. Despite sleeping 12-14 hours at night, her sleep was often interrupted with terrors/nightmares with themes of pursuit and abandonment. The night terrors were episodic occurring for two to three weeks every three to four months. During these episodes, she would awaken soaked in sweat.

### Family Psychiatric History

Unknown

### Behavior and Psychiatric History

Subject has always been full of energy physically and mentally. Her mother describes her as waking up “bursting with energy or angry and agitated.” Subject's mood is erratic and extreme. Some mornings stricken with rage she attacks her mother, other mornings Subject jumps up and down expressing how beautiful the day is. Her temperament is rarely even.

Her mood swings have been observed since 18 mos with the intensity and duration fluctuating every three to four months. Her daily mood variation is described as typically elated (goofy, hyperactive) for 2-3 hours, then switches to the opposite extreme of angry, irritable, and agitated for about 2 hours. She usually remains stable at school and playdates but afterwards alternates between extremes. Subject is in “constant motion”—restless, fidgety and easily distracted.

Subject is triggered into rage by the answer “no,” separation from her mother, loud noises, and the wind. She also has extreme difficulty with transitions, always in need of a transitional object and/or rituals. She also has difficulty with hoarding.

Subject has such difficulty with the answer “no” that in a fit of rage, she nearly ejected herself out of a moving taxi. She has also, unprovoked, physically attacked her parents, kicking and punching at their heads while they were in bed. While her parents restrain her, Subject thrashes and screams “don't hurt me, don't hurt me.” Her parents describe her voice in rages as distinctly different from that of normal. After her rages, Subject abruptly returns to normal as if nothing happened. When asked about her episodes of rage, Subject replies “nothing, nothing.

Subject also occasionally suffers from hallucinations (olfactory, visual) and delusions. She is quite fearful of others. One time when asked why she was screaming with reference to people outside, she replied “[they] are going to come kill us with a gun”.

### School History

During the first few months of school, Subject would rage everyday when being separated from her mother. However, mid-year Subject no longer raged during separation or at school. School officials describe her as charming, engaging, and funny. Subject has been described by her parents as smart, curious, and extremely social. She is in preschool with 10 peers and 2 teachers. Subject enjoys school; her mother describes it as offering her lots of freedom within the structure that she needs and craves.

### Ketamine Dosage

Subject is currently on seroquel—225 mg (50 mg time release mornings, 25 mg afternoon, 150 mg time release evenings), lithium 600 mg (300 mg mornings and evenings), and 2 intranasal puffs of ketamine.

### Response to Ketamine

After the use of Ketamine, Subject had quite notable changes in body temperature, speech, levels of fear, sleep, attention, aggression, and psychosis/delusions.

Subject's aggression has nearly disappeared, returning on occasion the few days prior to her next ketamine administration. Her delusions have also subsided with exception of the last few days of her ketamine cycle. Her hallucinations have gone into complete remission.

Her sleep has improved greatly. Before ketamine, Subject would awaken several times a night unable to return to sleep on her own. After ketamine, Subject has been able to most nights uninterrupted. If she awakens, she is able to self-sooth herself to sleep again. With ketamine, she reports “good dreams” have replaced her “bad dreams.”

Subject has been able to cope with transitions without the aid of transitional rituals and her dependence on transitional objects has decreased to the rare occasion. Her hoarding behavior has ceased.

Subject has experienced much improvement in her physical well-being. She no longer complains of night sweats and no longer awakens extremely thirsty. She has experienced improved coordination, no more motion sickness, and has expressed a newfound interest in grooming and exercise.

Subject's speech has also improved with much greater regularity in coherence, tempo, volume, and clarity. Her conversations have also grown to be more expressive.

What is claimed is:

1. A method for treating childhood-onset bipolar disorder in a subject in need thereof comprising administering a therapeutic-effective amount of ketamine.

2. The method of claim 1, wherein the subject is under the age of 18.

3. The method of claim 1, wherein the subject presents with Fear of Harm.

4. The method of claim 1, wherein the subject presents with at least one of the following symptoms: mood cycling (i.e., cycling between manic episodes, depressive episodes, and normal moods), obsessive fear of harm, severe aggression, territorial aggression, thermal dysregulation, night sweats, inability to fall asleep, inability to stay asleep, disorganized speech, rapid speech, loud speech, unclear speech, unusual speech timbre, disorganized thoughts, excessive ritualization, reliance on transitional objects, hoarding, extreme separation anxiety, hallucinations, delusions, and sweet cravings.

5. The method of claim 1, wherein the ketamine is racemic.

6. The method of claim 1, wherein the ketamine is (R)-ketamine.

7. The method of claim 1, wherein the ketamine is (S)-ketamine.

8. The method of claim 1, wherein the ketamine composition is administered nasally.

9. The method of claim 1, wherein the ketamine composition is administered orally.

10. The method of claim 1, wherein the therapeutically-effective amount of ketamine is about 0.01 to about 1 mg/kg of body weight.

11. The method of claim 1, wherein the therapeutically-effective amount of ketamine is about 0.05 to about 0.7 mg/kg of body weight.

12. Use of a therapeutic-effective amount of ketamine to treat childhood-onset bipolar disorder with Fear of Harm.

13. The use of claim 12, wherein the subject is under the age of 18.

14. The use of claim 12, wherein the subject presents with Fear of Harm.

15. The use of claim 12, wherein the subject presents with at least one of the following symptoms: mood cycling (i.e., cycling between manic episodes, depressive episodes, and normal moods), obsessive fear of harm, severe aggression, territorial aggression, thermal dysregulation, night sweats, inability to fall asleep, inability to stay asleep, disorganized speech, rapid speech, loud speech, unclear speech, unusual speech timbre, disorganized thoughts, excessive ritualization, reliance on transitional objects, hoarding, extreme separation anxiety, hallucinations, delusions, and sweet cravings.

16. The use of claim 12, wherein the ketamine is racemic.

17. The use of claim 12, wherein the ketamine is (R)-ketamine.

18. The use of claim 12, wherein the ketamine is (S)-ketamine.

19. The use of claim 12, wherein the ketamine composition is administered nasally.

20. The use of claim 12, wherein the ketamine composition is administered orally.

21. The use of claim 12, wherein the therapeutically-effective amount of ketamine is about 0.01 to about 1 mg/kg of body weight.

22. The use of claim 12, wherein the therapeutically-effective amount of ketamine is about 0.05 to about 0.7 mg/kg of body weight.
